(19) United States
(12) Patent Application Publication
(10) Pub. No.: US 2025/0152553 A1
(43) Pub. Date: May 15, 2025

(54) CARBAMOTHIOATE, AND CARBAMODITHIOATE DERIVATIVES OF MDMA, MDA OR ITS OPTICALLY ACTIVE (R)- OR (S)-MDMA AND MDA ISOMERS

(71) Applicant: EmpathBio, Inc., New York, NY (US)

(72) Inventors: Tanweer A. KHAN, Bridgewater, NJ (US); Glenn SHORT, Scituate, MA (US); Robert B. PERNI, Marlborough, MA (US)

(21) Appl. No.: 18/934,442

(22) Filed: Nov. 1, 2024

Related U.S. Application Data
(60) Provisional application No. 63/596,060, filed on Nov. 3, 2023.

(51) Int. Cl. A61K 31/36; C07D 317/58

(52) U.S. Cl. CPC A61K 31/36; C07D 317/58

(57) ABSTRACT

Provided herein are carbamothioate and carbamodithioate derivates of 3,4-methylenedioxymethamphetamine, (R)-3,4-methylenedioxymethamphetamine and (S)-3,4-methylenedioxymethamphetamine.

### CROSS-REFERENCE TO RELATED APPLICATION

This application claims priority from U.S. Provisional Application No. 63/596,060, filed Nov. 3, 2023, which is hereby incorporated by reference in its entirety.

### BACKGROUND

MDMA (3,4-methylenedioxymethamphetamine) is considered the prototype of a class of compounds called entactogens, which means “to touch within”, their main characteristic being their ability to increase feelings of love, empathy and closeness towards others. Structurally, MDMA is a ring-substituted phenethylamine with a chiral molecular center that gives rise to two stereoisomers: S-(+)-MDMA and R-(−)-MDMA. Typically, effects of the former resemble those of psychostimulants and are primarily mediated by dopaminergic and noradrenergic pathways, including increases in motor activity and euphoria, whereas the latter induces qualitative effects similar to classical psychedelics, such as ego-dissolution and perceptive alterations, mediated by serotonergic pathways, including direct 5-HT2A receptor agonism. The molecular mechanisms for these differences are supported by preclinical evidence and point to a higher therapeutic index for the R-enantiomer.

There remains a need for improved derivatives of MDMA (3,4-methylenedioxymethamphetamine) and pharmaceutically acceptable salts thereof and enantiomers thereof.

### SUMMARY

In an aspect, the present disclosure relates to 3,4-methylenedioxymethamphetamine (MDMA) derivatives, in particular, the present disclosure provides novel carbamothioate and carbamodithioate MDMA derivatives. In one embodiment the present disclosure provides a compound having the formula of Formula (VI)

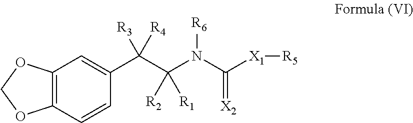

wherein X₁ and X₂ are independently O, S or N,
   R₁, R₂, R₃, and R₄ are independently hydrogen, deuterium, alkyl, deuterated alkyl, CH₂D, CHD₂, CD₃, cycloalkyl, halogen, heteroatom-substituted or halogen-substituted alkyl or cycloalkyl, aryl, or heteroaryl; and R₅ and R₆, are independently hydrogen, deuterium, methyl, ethyl, CH₂D, CHD₂, CD₃, or a C₁ to C₆ alkyl.

In some embodiments, X₂ is S. In some embodiments, X₂ is O. In some embodiments, X₁ and X₂ are S.

In some embodiments, R₁ is methyl and R₂, R₃, R₄, and R₆ are hydrogen. In some embodiments, R₅ is methyl. In some embodiments, R₆ is methyl. In some embodiments, R₁ and R₂ combine to form a 3-6 membered cycloalkyl.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₅, C₂₋₅, C₂₋₄, C₂₋₃, C₃₋₅C₃₋₅, C₃₋₄, C₄₋₅, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heterocyclyl” “heterocyclic ring” or “heterocycle” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In embodiments where “Ring A” is heterocyclyl, the heterocyclyl radical is a diradical. Unless stated otherwise specifically in the specification, a heterocyclyl group can be optionally substituted.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

The term “substituted” used herein means any of the groups described herein (e.g., alkyl, heterocyclyl, and/or heteroaryl) wherein at least one hydrogen atom is replaced by a bond to a non-hydrogen atoms such as, but not limited to: deuterium (i.e., “D”), a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

The term “carbamothioate” or “thiocarbamate” refers to a —N—(C═O)—S— or —N—(C═S)—O— moiety. In some embodiments, the moiety is a linkage between alkyl groups, deuterated alkyl groups, substituted groups, aryl groups, deuterated aryl groups, cycloalkyl groups, substituted cycloalkyl groups, or any combination of the groups described herein. [needed?]

The term “carbamodithioate” or “dithiocarbamate” refers to a —N—(C═S)—S— or —N—(C═S)—S— moiety. In some embodiments, the moiety is a linkage between alkyl groups, deuterated alkyl groups, substituted groups, aryl groups, deuterated aryl groups, cycloalkyl groups, substituted cycloalkyl groups, or any combination of the groups described herein.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, such as (R)- or (S)-. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Where a particular stereoisomer is expressly depicted, it is to be understood to refer to only that particular stereoisomer.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

As used herein, the symbol

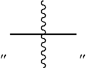

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

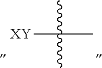

indicates that the chemical entity “XY” is bonded to another chemical entity via the point of attachment bond. Furthermore, the specific point of attachment to the non-depicted chemical entity can be specified by inference. For example, the compound CH₃—R^{3X}, wherein R^{3X} is H or

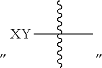

infers that when R^{3X} is “XY”, the point of attachment bond is the same bond as the bond by which R^{3X} is depicted as being bonded to CH₃.

### General Implementation

The present disclosure relates to 3,4-methylenedioxymethamphetamine (MDMA) derivatives, in particular, the present disclosure provides novel thiocarbamate MDMA derivatives. In one embodiment the present disclosure provides a compound having the formula of Formula (IV) Formula (VI)

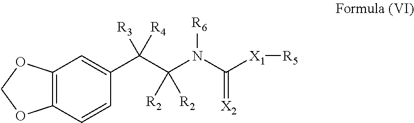

wherein X₁ and X₂ are independently O, S or N,
R₁, R₂, R₃, and R₄ are independently hydrogen, deuterium, alkyl, deuterated alkyl, CH₂D, CHD₂, CD₃, cycloalkyl, halogen, heteroatom substituted or halogen substituted alkyl or cycloalkyl, aryl, or heteroaryl; and R₅ and R₆, are independently hydrogen, deuterium, methyl, ethyl, CH₂D, CHD₂, CD₃, or a C₁ to C₆ alkyl.

In some embodiments, X₂ is S. In some embodiments, X₂ is O. In some embodiments, X₁ and X₂ are S.

In some embodiments, R₁ is methyl and R₂, R₃, R₄, and R₆ are hydrogen. In some embodiments, R₅ is methyl. In some embodiments, R₆ is methyl. In some embodiments, R₁ and R₂ combine to form a 3-6 membered cycloalkyl.

In some embodiments, R₅ is propyl, butyl, a 3-7 membered saturated ring, a 5 membered ring, a 6 membered ring, a phenyl, a benzyl, or a CH₂-heteroaryl. In some embodiments, R₅ can be a C₁ to C₆ alkyl optionally substituted with a halogen atom.

In some embodiments, the compounds of Formula (VI) comprise the compounds of Table 1:

TABLE 1

| S.N. | Structure | Name |
|---|---|---|
| 1 | 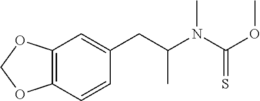 [SMILES: COC(=S)N(C)C(C)Cc1ccc2c(c1)OCO2] | O-methyl (1-(benzo[d][1,3]dioxol-5- yl)propan-2-yl)(methyl)carbamothioate |
| 2 | 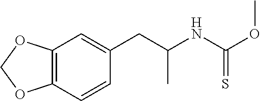 [SMILES: COC(=S)NC(C)Cc1ccc2c(c1)OCO2] | O-methyl (1-(benzo[d][1,3]dioxol-5- yl)propan-2-yl)carbamothioate |
| 3 | 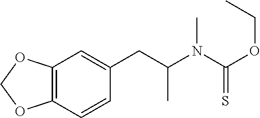 [SMILES: CCOC(=S)N(C)C(C)Cc1ccc2c(c1)OCO2] | O-ethyl (1-(benzo[d][1,3]dioxol-5- yl)propan-2-yl)(methyl)carbamothioate |
| 4 | 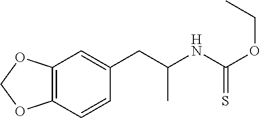 [SMILES: CCOC(=S)NC(C)Cc1ccc2c(c1)OCO2] | O-ethyl (1-(benzo[d][1,3]dioxol-5- yl)propan-2-yl)carbamothioate |
| 5 | 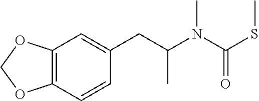 [SMILES: CSC(=O)N(C)C(C)Cc1ccc2c(c1)OCO2] | S-methyl (1-(benzo[d][1,3]dioxol-5- yl)propan-2-yl)(methyl)carbamothioate |
| 6 | 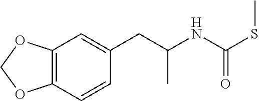 [SMILES: CSC(=O)NC(C)Cc1ccc2c(c1)OCO2] | S-methyl (1-(benzo[d][1,3]dioxol-5- yl)propan-2-yl)carbamothioate |
| 7 | 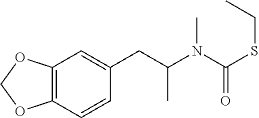 [SMILES: CCSC(=O)N(C)C(C)Cc1ccc2c(c1)OCO2] | S-ethyl (1-(benzo[d][1,3]dioxol-5- yl)propan-2-yl)(methyl)carbamothioate |
| 8 | 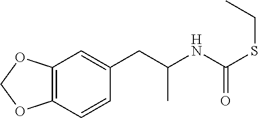 [SMILES: CCSC(=O)NC(C)Cc1ccc2c(c1)OCO2] | S-ethyl (1-(benzo[d][1,3]dioxol-5- yl)propan-2-yl)carbamothioate |
| 9 | 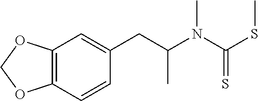 [SMILES: CSC(=S)N(C)C(C)Cc1ccc2c(c1)OCO2] | methyl (1-(benzo[d][1,3]dioxol-5- yl)propan-2-yl)(methyl)carbamodithioate |
| 10 | 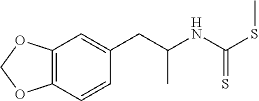 [SMILES: CSC(=S)NC(C)Cc1ccc2c(c1)OCO2] | methyl (1-(benzo[d][1,3]dioxol-5- yl)propan-2-yl)carbamodithioate |
| 11 | 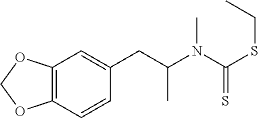 [SMILES: CCSC(=S)N(C)C(C)Cc1ccc2c(c1)OCO2] | ethyl (1-(benzo[d][1,3]dioxol-5-yl)propan- 2-yl)(methyl)carbamodithioate |
| 12 | 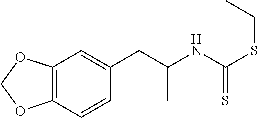 [SMILES: CCSC(=S)NC(C)Cc1ccc2c(c1)OCO2] | ethyl (1-(benzo[d][1,3]dioxol-5-yl)propan- 2-yl)carbamodithioate |
| 13 | 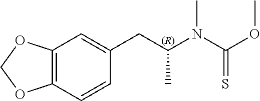 | O-methyl (R)-(1-(benzo[d][1,3]dioxol-5- yl)propan-2-yl)(methyl)carbamothioate |
| 14 | 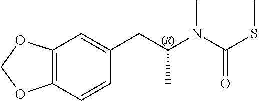 | S-methyl (R)-(1-(benzo[d][1,3]dioxol-5- yl)propan-2-yl)(methyl)carbamothioate |
| 15 | 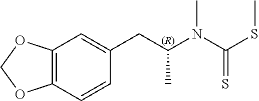 | methyl (R)-(1-(benzo[d][1,3]dioxol-5- yl)propan-2-yl)(methyl)carbamodithioate |
| 16 | 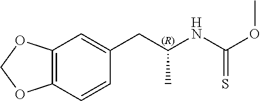 | O-methyl (R)-(1-(benzo[d][1,3]dioxol-5- yl)propan-2-yl)carbamothioate |
| 16 | 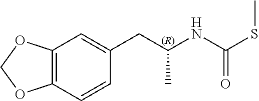 | S-methyl (R)-(1-(benzo[d][1,3]dioxol-5- yl)propan-2-yl)carbamothioate |
| 18 | 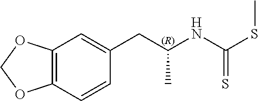 | methyl (R)-(1-(benzo[d][1,3]dioxol-5- yl)propan-2-yl)carbamodithioate |

In some embodiments, the compounds of Formula (VI) comprise the compounds of:

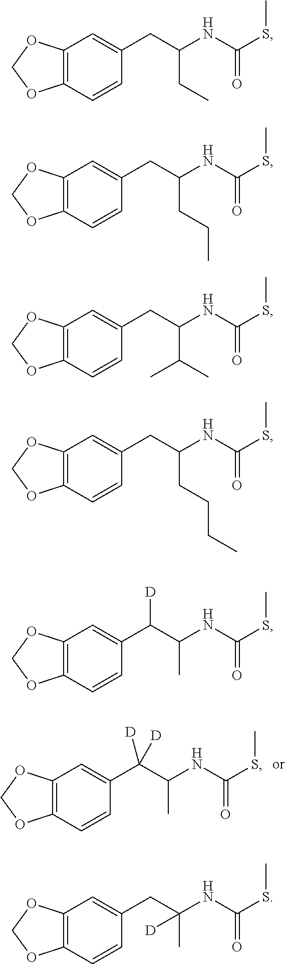

In some embodiments, the compounds of Formula (VI) comprise the compounds of:

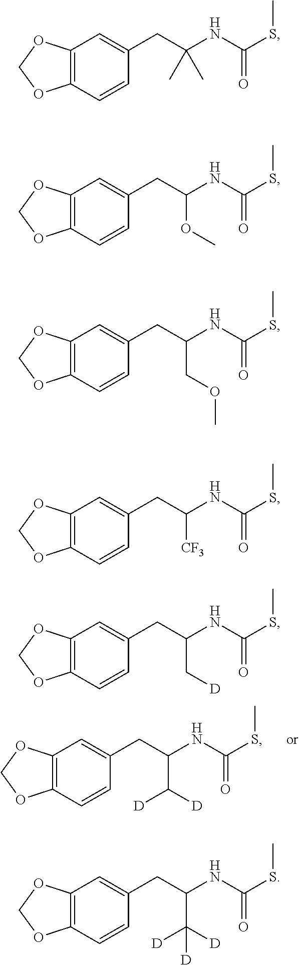

In some embodiments, the compounds of Formula (VI) comprise the compounds of:

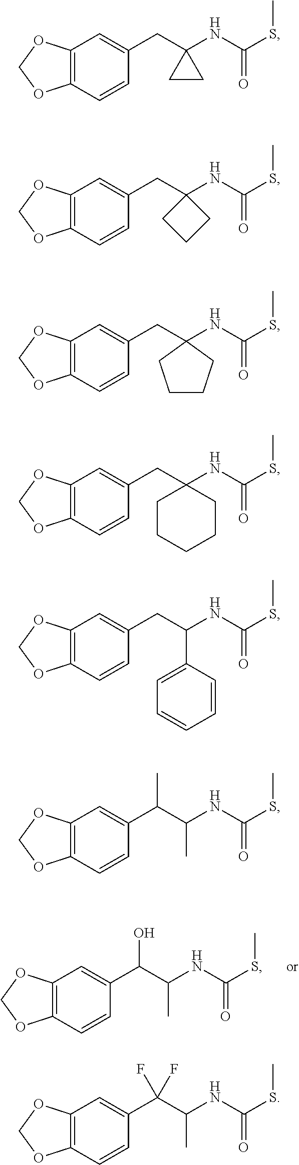

### Methods

In an aspect, the methods described herein provide high purity, 3,4-methylenedioxymethamphetamine (MDMA) in a high yielding 3-step process, starting from readily available and inexpensive starting materials (e.g., 5-bromobenzo[d][1,3]dioxole (3) and alaninol). In an aspect, the disclosure provides a process for preparation of racemic 3,4-methylenedioxymethamphetamine or a pharmaceutically acceptable salt thereof.

In an aspect, the disclosure provides a process for the preparation of (R)- or (S)-3,4-methylenedioxymethamphetamine, or a pharmaceutically acceptable salt thereof.

In embodiments, the methods described herein provide high purity, enantiopure (R) or (S) 3,4-methylenedioxymethamphetamine (MDMA) in a high yielding 3-step process, starting from readily available and inexpensive starting materials (e.g., 5-bromobenzo[d][1,3]dioxole (3) and D-alaninol (1)). By starting with a chiral pool starting materials, such as (D-alaniol (1)), enantiopure (R)-MDMA can be prepared without the need for expensive and wasteful chiral ligands, chiral auxiliaries, or diastereomeric salt resolutions, and provide MDMA with higher optical purity (e.g., 99.5% ee or greater, or 99.9% ee) than other routes, which give lower selectivity and require enantiomeric enrichment by purification/crystallization. By installing a Boc group as both a nitrogen protecting group and a masked methyl equivalent, the need for a methylation reaction is avoided, which often uses GTI alkylating reagents like methyl iodide or dimethylsulfate. It also cleanly installs the single methyl group and avoids the potential of over-alkylation and provides enantioenriched (R)-MDMA in a streamlined process, providing significant improvements on prior syntheses which are more complex and/or require synthetic longer routes see e.g., ACS Chem Neurosci. 2018 Oct. 17; 9(10): 2408-2427, FIG. 3. The unnatural enantiomer of (1) (L-alaninol) can be used by the same process to prepare (S)-MDMA.

### Preparation of 3,4-Methylenedioxymethamphetamine

In an aspect, the disclosure provides a process for the preparation of 3,4-methylenedioxymethamphetamine, or a pharmaceutically acceptable salt thereof, the process comprising:
i) preparing an organometallic reagent from a compound of Formula (I):

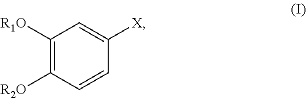

wherein X is a halogen; R₁ is a protecting group, R₂ is a protecting group or R₁ and R₂ together with the atoms to which they are attached form a 5-membered heterocycle;
ii) reacting the organometallic reagent of step i) with a compound of Formula (IIb):

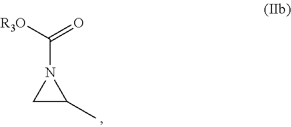

wherein R₃ is alkyl; and
iii) converting the product of step ii) to 3,4-methylenedioxymethamphetamine.

In embodiments, the compound of Formula (IIb) is a racemate.

In embodiments of the processes provided herein for the preparation of 3,4-methylenedioxymethamphetamine, the process comprises i) preparing an organometallic reagent from a compound of Formula (I):

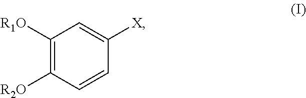

wherein X is a halogen; R₁ is a protecting group, R₂ is a protecting group or R₁ and R₂ together with the atoms to which they are attached form a 5-membered heterocycle.

In some embodiments of any one of the processes for the preparation of 3,4-methylenedioxymethamphetamine described herein, R₁ and R₂ together with the atoms to which they are attached form a 5-membered heterocycle.

In some embodiments of the process for the preparation of 3,4-methylenedioxymethamphetamine, the compound of Formula (I) is a compound of Formula (Ia):

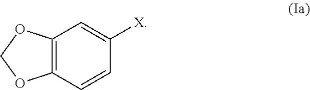

In embodiments of the compounds of Formula (I) or (Ia), X is Cl, Br, or I. In some embodiments, X is Cl. In some embodiments, X is Br. In some embodiments, X is I.

In some embodiments of the process for the preparation of 3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, or 2-methyltetrahydrofuran. In some embodiments, the solvent is heated e.g., to 50-70° C., 60-70° C., or 60-66° C. In some embodiments, the solvent is heated to reflux. In some embodiments, the solvent is THF and the THF is heated to reflux.

In some embodiments of the process for the preparation of 3,4-methylenedioxymethamphetamine, step i) further comprises adding a copper (I) salt (e.g., CuI, CuCl, or CuBr—SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In embodiments of the process for the preparation of 3,4-methylenedioxymethamphetamine, step ii) comprises reacting the organometallic reagent of step i) with a compound of Formula (IIb):

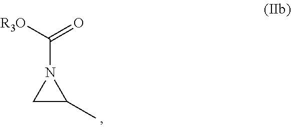

wherein R₃ is alkyl. In some embodiments, step ii) comprises adding a compound of Formula (IIb) to the cooled solution of organometallic reagent from step i).

In some embodiments of the process for the preparation of 3,4-methylenedioxymethamphetamine, R₃ is C₁₋₆ alkyl. In some embodiments, R₃ is C₁₋₄ alkyl. In some embodiments, R₃ is tert-butyl.

In some embodiments of the process for the preparation of 3,4-methylenedioxymethamphetamine, the product of step ii) is a compound of Formula (IIIb):

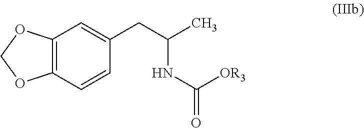

wherein R₃ is defined herein.

In some embodiments of the process for the preparation of 3,4-methylenedioxymethamphetamine, the product of step ii) is a compound of Formula (IIIc):

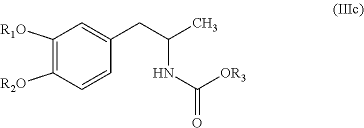

wherein R₁, R₂, and R₃ are defined herein.

In some embodiments of the process for the preparation of 3,4-methylenedioxymethamphetamine, step iii) comprises reacting a group of Formula (IVb) with a reducing agent to provide a group of Formula (Vb), or a pharmaceutically acceptable salt thereof:

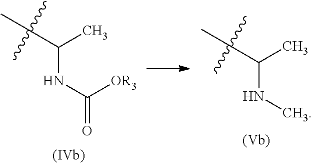

In some embodiments, step iii) comprises reacting a group of Formula (IVb) with a reducing agent to provide a group of Formula (Vb), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of 3,4-methylenedioxymethamphetamine, the reducing agent in step iii) is a hydride reducing agent.

In some embodiments, the reducing agent in step iii) is lithium aluminum hydride.

In some embodiments of the process for the preparation of 3,4-methylenedioxymethamphetamine, the process provides 3,4-methylenedioxymethamphetamine, or a pharmaceutically acceptable salt thereof in racemic form.

In some embodiments of the process for the preparation of 3,4-methylenedioxymethamphetamine, the 3,4-methylenedioxymethamphetamine, or a pharmaceutically acceptable salt thereof has a chemical purity of greater than about 95%, greater than about 98%, or greater than about 99% by HPLC.

In some embodiments, the present disclosure provides 3,4-methylenedioxymethamphetamine, or a pharmaceutically acceptable salt prepared by a process described herein.

### Preparation of (S)-3,4-methylenedioxymethamphetamine

In an aspect, the disclosure provides a process for the preparation of (S)-3,4-methylenedioxymethamphetamine, or a pharmaceutically acceptable salt thereof, the process comprising:
i) preparing an organometallic reagent from a compound of Formula (I):

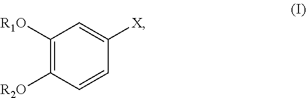

wherein X is a halogen; R₁ is a protecting group, R₂ is a protecting group or R₁ and R₂ together with the atoms to which they are attached form a 5-membered heterocycle;
ii) reacting the organometallic reagent of step i) with a compound of Formula (II):

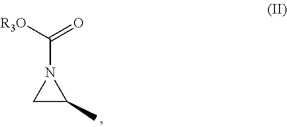

wherein R₃ is alkyl; and
iii) converting the product of step ii) to (S)-3,4-methylenedioxymethamphetamine.

In embodiments of the processes provided herein for the preparation of (S)-3,4-methylenedioxymethamphetamine, the process comprises i) preparing an organometallic reagent from a compound of Formula (I):

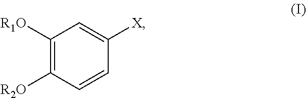

wherein X is a halogen; R₁ is a protecting group, R₂ is a protecting group or R₁ and R₂ together with the atoms to which they are attached form a 5-membered heterocycle.

In some embodiments of any one of the processes for the preparation of (S)-3,4-methylenedioxymethamphetamine described herein, R₁ and R₂ together with the atoms to which they are attached form a 5-membered heterocycle.

In some embodiments of the process for the preparation of (S)-3,4-methylenedioxymethamphetamine, the compound of Formula (I) is a compound of Formula (Ia):

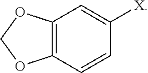

In embodiments of the compounds of Formula (I) or (Ia), X is Cl, Br, or I. In some embodiments, X is Cl. In some embodiments, X is Br. In some embodiments, X is I.

In some embodiments of the process for the preparation of (S)-3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C., or 60-70° C., or 60-66° C. In some embodiments, the solvent is heated to reflux. In some embodiments, the solvent is THF and the THF is heated to reflux.

In some embodiments of the process for the preparation of (S)-3,4-methylenedioxymethamphetamine, step i) further comprises adding a copper (I) salt (e.g., CuI, CuCl, CuBr—SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyaphetamine.

In embodiments of the process for the preparation of (S)-3,4-methylenedioxymethamphetamine, step ii) comprises reacting the organometallic reagent of step i) with a compound of Formula (II):

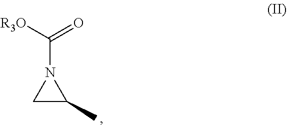

wherein R₃ is alkyl. In some embodiments, step ii) comprises adding a compound of Formula (II) to the cooled solution of organometallic reagent from step i).

In some embodiments of the process for the preparation of (S)-3,4-methylenedioxymethamphetamine, R₃ is C₁₋₆ alkyl. In some embodiments, R₃ is C₁₋₄ alkyl. R₃ is tert-butyl.

In some embodiments of the process for the preparation of (S)-3,4-methylenedioxymethamphetamine, the product of step ii) is a compound of Formula (III):

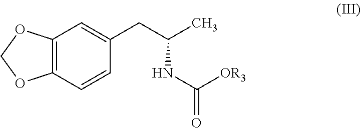

wherein R₃ is defined herein.

In some embodiments of the process for the preparation of (S)-3,4-methylenedioxymethamphetamine, the product of step ii) is a compound of Formula (IIIa):

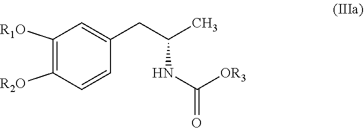

wherein R₁, R₂, and R₃ are defined herein.

In some embodiments of the process for the preparation of (S)-3,4-methylenedioxymethamphetamine, step iii) comprises reacting a group of Formula (IV) with a reducing agent to provide a group of Formula (V), or a pharmaceutically acceptable salt thereof:

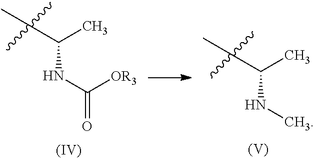

In some embodiments, step iii) comprises reacting a group of Formula (IV) with a reducing agent to provide a group of Formula (V), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of (S)-3,4-methylenedioxymethamphetamine, the reducing agent in step iii) is a hydride reducing agent. In some embodiments, the reducing agent in step iii) is lithium aluminum hydride.

In some embodiments of the process for the preparation of (S)-3,4-methylenedioxymethamphetamine, the process provides (S)-3,4-methylenedioxymethamphetamine, or a pharmaceutically acceptable salt thereof in substantially optically pure form. In some embodiments, the process for the preparation of (S)-3,4-methylenedioxymethamphetamine, the process provides (S)-3,4-methylenedioxymethamphetamine, or a pharmaceutically acceptable salt in an enantiomeric excess of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (S)-3,4-methylenedioxymethamphetamine, the process provides (S)-3,4-methylenedioxymethamphetamine, or a pharmaceutically acceptable salt in an enantiomeric excess of a least 99.5%. In some embodiments, ee is measured by chiral HPLC.

In some embodiments of the process for the preparation of (S)-3,4-methylenedioxymethamphetamine, the (S)-3,4-methylenedioxymethamphetamine, or a pharmaceutically acceptable salt thereof has a chemical purity of greater than about 95%, greater than about 98%, or greater than about 99% by HPLC.

In some embodiments, the present disclosure provides (S)-3,4-methylenedioxymethamphetamine, or a pharmaceutically acceptable salt prepared by a process described herein.

### Preparation of (R)-3,4-methylenedioxymethamphetamine

In an aspect, the disclosure provides a process or the preparation of (R)-3,4-methylenedioxymethamphetamine, or a pharmaceutically acceptable salt thereof, the method comprising:
i) preparing an organometallic reagent from a compound of Formula (I):

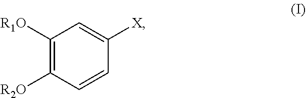

wherein X is a halogen; R₁ is a protecting group, R₂ is a protecting group or R₁ and R₂ together with the atoms to which they are attached form a 5-membered heterocycle; ii) reacting the organometallic reagent of step i) with a compound of Formula (IIa):

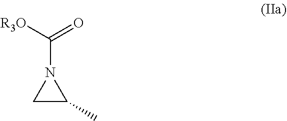

wherein R₃ is alkyl; and
iii) converting the product of step ii) to (R)-3,4-methylenedioxymethamphetamine.

In embodiments of the processes provided herein for the preparation of (R)-3,4-methylenedioxymethamphetamine, the process comprises i) preparing an organometallic reagent from a compound of Formula (I):

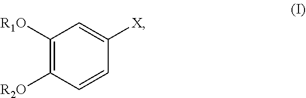

wherein X is a halogen; R₁ is a protecting group, R₂ is a protecting group or R₁ and R₂ together with the atoms to which they are attached form a 5-membered heterocycle.

In some embodiments of any one of the processes for the preparation of (R)-3,4-methylenedioxymethamphetamine described herein, R₁ and R₂ together with the atoms to which they are attached form a 5-membered heterocycle.

In some embodiments of the process for the preparation of (R)-3,4-methylenedioxymethamphetamine, the compound of Formula (I) is a compound of Formula (Ia):

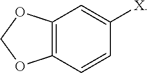

In embodiments of the compounds of Formula (I) or (Ia), X is Cl, Br, or I. In some embodiments, X is Cl. In some embodiments, X is Br. In some embodiments, X is I.

In some embodiments of the process for the preparation of (R)-3,4-methylenedioxymethamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C. In some embodiments, the solvent is THF and the THF is heated to reflux.

In some embodiments of the process for the preparation of (R)-3,4-methylenedioxymethamphetamine, step i) further comprises adding a copper (I) salt (e.g., CuI, CuCl, CuBr—SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In some embodiments of the process for the preparation of (R)-3,4-methylenedioxymethamphetamine, step ii) comprises reacting the organometallic reagent of step i) with a compound of Formula (IIa):

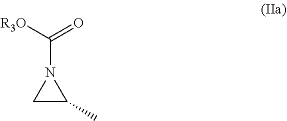

wherein R₃ is alkyl. In some embodiments, step ii) comprises adding a compound of Formula (IIa) to the cooled solution of organometallic reagent from step i).

In some embodiments of the process for the preparation of (R)-3,4-methylenedioxymethamphetamine, R₃ is C₁₋₆ alkyl. In some embodiments, R₃ is C₁₋₄ alkyl. R₃ is tert-butyl.

In some embodiments of the process for the preparation of (R)-3,4-methylenedioxymethamphetamine, the product of step ii) is a compound of Formula (IIIb):

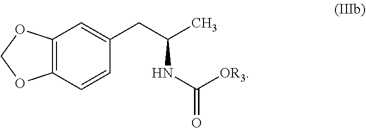

In some embodiments of the process for the preparation of (R)-3,4-methylenedioxymethamphetamine, the product of step ii) is a compound of Formula (IIIb′):

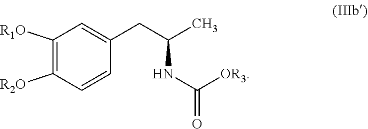

In some embodiments of the process for the preparation of (R)-3,4-methylenedioxymethamphetamine, step iii) comprises reacting a group of Formula (IVa) with a reducing agent to provide a group of Formula (Va), or a pharmaceutically acceptable salt thereof:

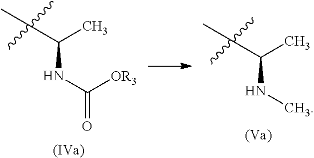

In some embodiments, step iii) comprises reacting a group of Formula (IV) with a reducing agent to provide a group of Formula (V), or a pharmaceutically acceptable salt thereof in the presence of a solvent, for example an ether solvent such as tetrahydrofuran, diethyl ether, 2-methyltetrahydrofuran.

In some embodiments of the process for the preparation of (R)-3,4-methylenedioxymethamphetamine, the reducing agent in step iii) is a hydride reducing agent. In some embodiments, the reducing agent in step iii) is lithium aluminum hydride.

In some embodiments of the process for the preparation of (R)-3,4-methylenedioxymethamphetamine, the process provides (R)-3,4-methylenedioxymethamphetamine, or a pharmaceutically acceptable salt thereof in substantially optically pure form. In some embodiments, the process for the preparation of (R)-3,4-methylenedioxymethamphetamine, the process provides (R)-3,4-methylenedioxymethamphetamine, or a pharmaceutically acceptable salt in an enantiomeric excess of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (R)-3,4-methylenedioxymethamphetamine, the process provides (R)-3,4-methylenedioxymethamphetamine, or a pharmaceutically acceptable salt in an enantiomeric excess of a least 99.5%. In some embodiments, ee is measured by chiral HPLC.

In some embodiments of the process for the preparation of (R)-3,4-methylenedioxymethamphetamine, the (R)-3,4-methylenedioxymethamphetamine, or a pharmaceutically acceptable salt thereof has a chemical purity of greater than about 95%, greater than about 98%, or greater than about 99% by HPLC.

In some embodiments, the present disclosure provides (R)-3,4-methylenedioxymethamphetamine, or a pharmaceutically acceptable salt prepared by a process described herein.

Preparation of Carbamothioate and Carbamodithioate Derivatives of MDMA, MDA or it's Optically Active (R)- or (S)-MDMA and MDA Isomers.

In embodiments, Compounds 1, 2, 3, and 4 of Table 1 can be prepared from Scheme (VII).

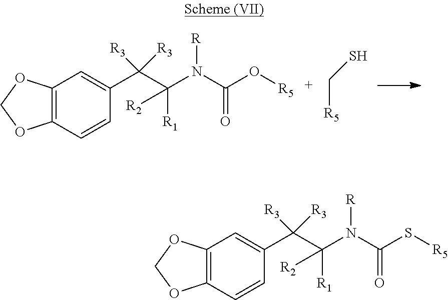

In embodiments a coupling reaction comprising the use of 2-Chloropyridine, Trifluoromethanesulfonic anhydride as reagents and dichloromethane as a solvent at room temperature is employed. For example, a coupling reaction as described by Kim, Hee-Kwon; et al, One-pot Synthesis of Carbamates and Thiocarbamates from Boc-protected Amines. Tetrahedron Letters (2016), 57(44), 4890-4892.

Compounds 1, 2, 3, and 4 of Table 1 can be prepared from Scheme (VIII).

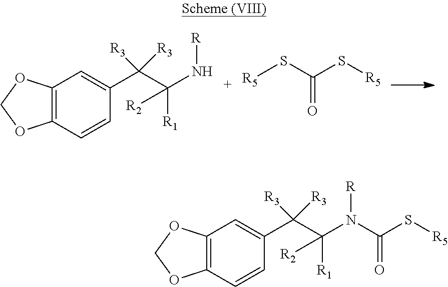

In embodiments, a coupling reaction comprising the use of water as a solvent at 20-25° C. is employed. For example, a coupling reaction as described by Artuso. Emma; et al, A general, facile, and safe procedure for the preparation of S-methyl N-alkylthiocarbamates by methylthiocarbonylation of primary aliphatic amines with S,S-dimethyl dithiocarbonate. Synthesis (2007), (7), 1096-1102.

Compounds 9, 10, 11, and 12 of Table 1 can be prepared from Scheme (IX).

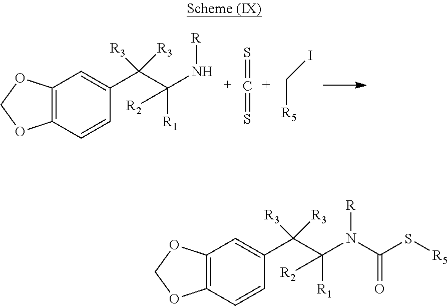

In embodiments, the coupling reaction comprises a first step of tripotassium phosphate as a reagent with acetone as a solvent and a second step of hydrochloric acid as a reagent with 1,4-diocane and water as solvents. For example, a coupling reaction as described by Ware, R. W.; et al, Evaluation of new 1-thiocitrulline derivatives as inhibitors of nitric oxide synthase. Bioorganic & Medical Chemistry Letters (2000), 10(24), 2779-2781.

Enantiomeric pure compounds were prepared by chiral resolution and chiral chromatographic purification.

### Numbered Embodiments

1. A compound of Formula (VI):

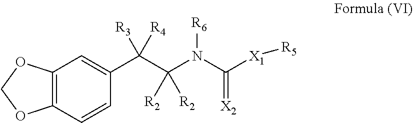

wherein X₁ and X₂ are independently O, S, or N;
R₁, R₂, R₃, and R₄ are independently hydrogen, deuterium, alkyl, deuterated alkyl, CH₂D, CHD₂, CD₃, cycloalkyl, halogen, heteroatom substituted or halogen substituted alkyl or cycloalkyl, aryl, or heteroaryl; and
R₅ and R₆, are independently hydrogen, deuterium, methyl, ethyl, CH₂D, CHD₂, CD₃, or a C₁ to C₆ alkyl.

2. The compound of embodiment 1, wherein X₂ is S.

3. The compound of embodiment 1 or embodiment 2, wherein X₁ is O.

4. The compound of embodiment 1 or embodiment 2, wherein X₁ is S.

5. The compound of any of embodiments 1-4, wherein R₂, R₃, R₄, and R₆ are hydrogen or deuterium.

6. The compound of any of embodiments 1-5, wherein R₁ is methyl.

7. The compound of any of embodiments 1-6, wherein R₅ is methyl.

8. The compound of any of embodiments 1-5, wherein R₁ is ethyl.

9. The compound of any of embodiments 1-5, wherein R₁ is propyl.

10. The compound of embodiment 9, wherein R₁ is isopropyl.

11. The compound of any of embodiments 1-5, wherein R₅ is ethyl.

12. The compound of any of embodiments 1-11, wherein R₆ is methyl.

13. The compound of any of embodiments 1-4, 7, 11 or 12, wherein R₁ and R₂ combine to form a 3-6 membered cycloalkyl.

14. The compound of embodiment 13, wherein R₁ and R₂ combine to form a 3 membered ring.

15. The compound of embodiment 13, wherein R₁ and R₂ combine to form a 4 membered ring.

16. The compound of embodiment 13, wherein R₁ and R₂ combine to form a 5 membered ring.

17. The compound of embodiment 13, wherein R₁ and R₂ combine to form a 6 membered ring.

18. The compound of any of embodiments 1-4, 7, 11, or 12, wherein R₁ is a halogenated alkyl.

19. The compound of any of embodiments 1-4 and 6-18, wherein R₃ and R₄ are halogens.

20. The compound of any of embodiments 1-4, 7, 11, or 12, wherein R₁ is an ether.

21. The compound of any of embodiments 1-20, wherein the compound is an R enantiomer.

22. The compound of any of embodiments 1-20, wherein the compound is an S enantiomer.

23. The compound of any of embodiments 1-22, wherein the compound persists through a first pass metabolism of a mammal.

24. The compound of any of embodiments 1-23, wherein the compound acts as a partial agonist of a CNS serotonin receptor.

25. A composition comprising a pharmaceutical carrier and a compound of Formula (VI):

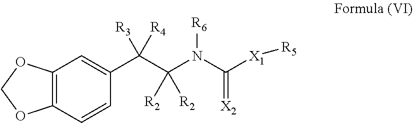

wherein X₁ and X₂ are independently O, S, or N;
R₁, R₂, R₃, and R₄ are independently hydrogen, deuterium, alkyl, deuterated alkyl, CH₂D, CHD₂, CD₃, cycloalkyl, halogen, heteroatom substituted or halogen substituted alkyl or cycloalkyl, aryl, or heteroaryl; and
R₅ and R₆, are independently hydrogen, deuterium, methyl, ethyl, CH₂D, CHD₂, CD₃, or a C₁ to C₆ alkyl.

26. The composition of embodiment 25, wherein X₂ is S.

27. The composition of embodiment 25 or 26, wherein X₁ is O.

28. The composition of embodiment 25 or 26, wherein X₁ is S.

29. The composition of any of embodiments 25-28, wherein R₂, R₃, R₄, and R₆ are hydrogen or deuterium.

30. The composition of any of embodiments 25-29, wherein R₁ is methyl.

31. The composition of any of embodiments 25-30, wherein R₅ is methyl.

32. The composition of any of embodiments 25-29, wherein R₁ is ethyl.

33. The composition of any of embodiments 25-29, wherein R₁ is propyl.

34. The composition of embodiment 33, wherein R₁ is isopropyl.

35. The composition of any of embodiments 25-30, or 32-34, wherein R₅ is ethyl.

36. The composition of any of embodiments 25-35, wherein R₆ is methyl.

37. The composition of any of embodiments 25-29, wherein R₁ and R₂ combine to form a 3-6 membered cycloalkyl.

38. The composition of embodiment 37, wherein R₁ and R₂ combine to form a 3 membered ring.

39. The composition of embodiment 37, wherein R₁ and R₂ combine to form a 4 membered ring.

40. The composition of embodiment 37, wherein R₁ and R₂ combine to form a 5 membered ring.

41. The composition of embodiment 37, wherein R₁ and R₂ combine to form a 6 membered ring.

42. The composition of any of embodiments 25-29, wherein R₁ is a halogenated alkyl.

43. The composition of any of embodiments 25-28 or 30-42, wherein R₃ and R₄ are halogens.

44. The composition of any of embodiments 25-29 or 43, wherein R₁ is an ether.

45. The composition of any of embodiments 25-44, wherein the compound is an R enantiomer.

46. The composition of any of embodiments 25-44, wherein the compound is an S enantiomer.

47. The composition of any of embodiments 25-46, wherein the compound persists through a first pass metabolism of a mammal.

48. The composition of any of embodiments 25-47, wherein the compound acts as a partial agonist of a CNS serotonin receptor.

49. The composition of any of embodiments 25-48, wherein the pharmaceutical carrier is a bulking agent.

### Examples

Compounds of the present disclosure can be synthesized using the following exemplary methods or other methods that are known to those skilled in the art.

General reaction conditions are provided, and reaction products can be purified by known methods including silica gel chromatography using various organic solvents such as hexane, dichloromethane, ethyl acetate, methanol and the like or preparative reverse phase high pressure liquid chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### General Synthesis of (R)-3,4-methylenedioxymethamphetamine (MDMA)

(R)-3,4-methylenedioxymethamphetamine (MDMA) can be synthesized according to Scheme 1, below.

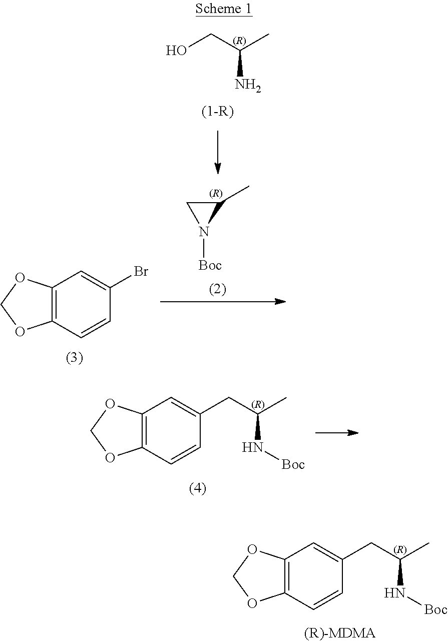

### Step 1: Synthesis of tert-butyl (R)-2-methylaziridine-1-carboxylate (2)

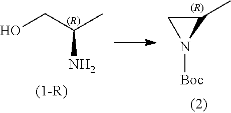

D-alaninol (1) is protected with di-tert-butyl decarbonate (Boc anhydride) in organic solvent (e.g., halogenated solvent such as dichloromethane (DCM), ether solvent such as tetrahydrofuran (THF) or methyl-tetrahydrofuran (Me-THF)) e.g. at <about 10° C. to ambient temperature, over a few hours. To the protected D-alaninol is then added 4-toluenesulfonyl chloride (TsCl), followed by potassium hydroxide (KOH), which installs the tosyl group on the alcohol, and deprotonates the carbamate nitrogen, facilitating aziridine formation. The enantiopure aziridine (2) is then isolated and purified, e.g., by chromatography on SiO₂ or by distillation.

### Step 2: Synthesis of tert-butyl (R)-(1-(benzo[d][1,3]dioxol-5-yl)propan-2-yl)carbamate (4)

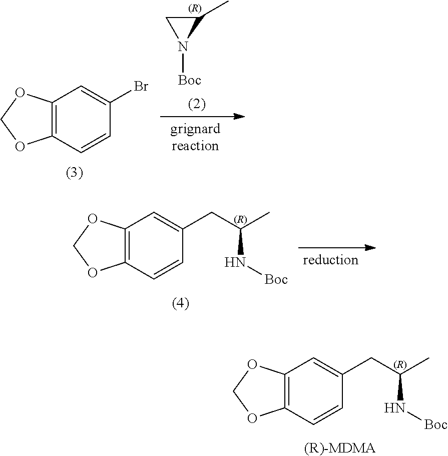

In a second step, the Grignard reagent of 5-bromobenzo[d][1,3]dioxole (3) is generated by treatment of 5-bromobenzo[d][1,3]dioxole (3) with magnesium, optionally in the presence of I₂, in organic solvent (e.g., ether solvent such as tetrahydrofuran (THF) or methyl-tetrahydrofuran (Me-THF)) and the mixture heated, e.g., between about 40° C. to about 70° C., or about 50° C. to about 60° C. After formation of the Grignard species, the solution is cooled (e.g., to about 0° C. or less) and Copper (I) salt e.g., CuI or CuBr·SMe₂ is added. The previously isolated Boc-aziridine (2) is then added to the cooled solution of cuprate and stirred until the reaction is complete. The reaction is then quenched with an aqueous solution e.g., with NH₄Cl and the organic phase extracted with organic solvent and purified by chromatography on SiO₂, or by crystallization.

### Step 3: Synthesis of (R)-3,4-methylenedioxymethamphetamine (MDMA)

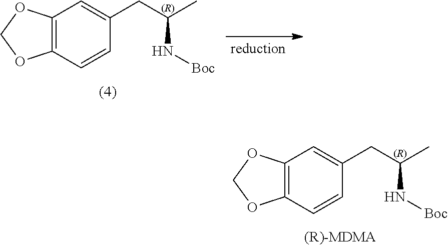

The Boc-MDA (4) is then dissolved in organic solvent (e.g., in an ether solvent such as tetrahydrofuran, (THF) or 2-methyltetrahydrofuran (Me-THF)), cooled and treated with lithium aluminum hydride (LiAlH4) to reduce the carbamate to the methylamine. In some embodiments, the solution is heated (e.g., to reflux) to reduce the carbamate to the methylamine. The reaction is quenched and worked-up via the Fieser method with water, NaOH and methyl tert-butyl ether. The crude MDMA free base can then be isolated e.g., by crystallization, or converted into the HCl salt using a solution of HCl.

(S)-3,4-methylenedioxymethamphetamine (MDMA) can be synthesized according to Scheme 1 employing L-alaninol instead of D-alaninol.

### Synthesis of Representative Compounds, 2, 4, 6, 8, 10 and 12.

These prodrugs were made via following three steps (scheme 1).

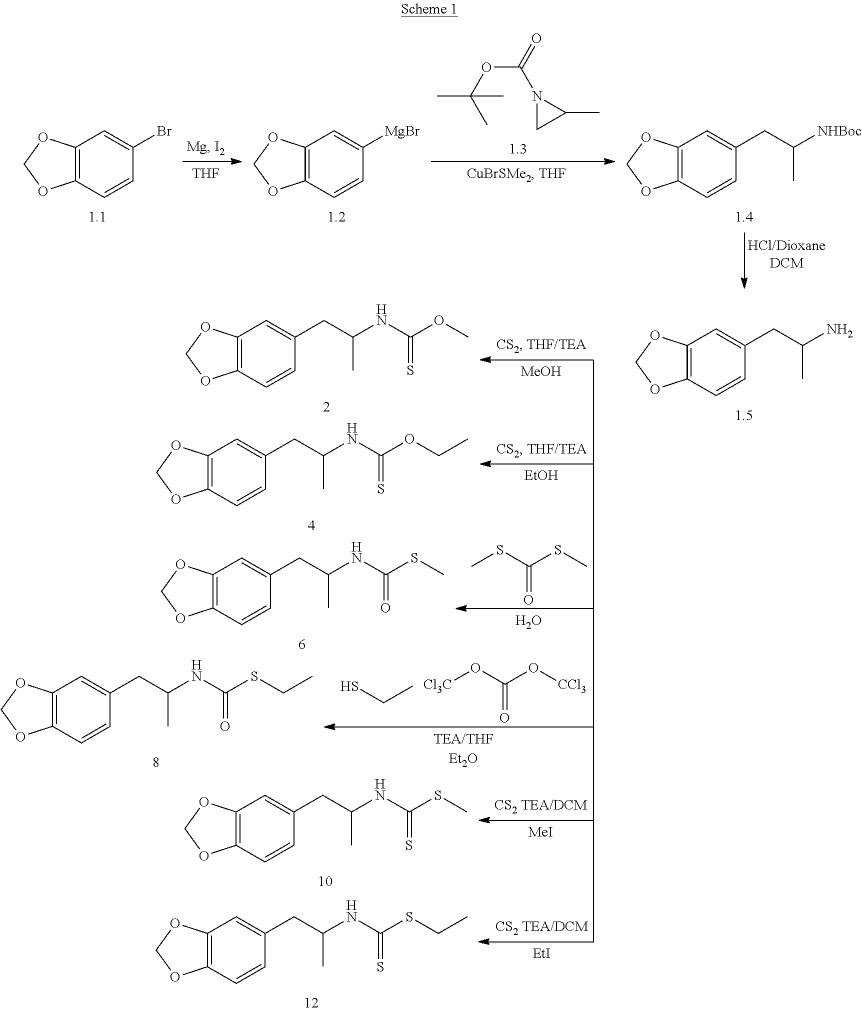

### Experimentals

### Synthesis of Intermediate 1.5.

### Step 1. 5-(bromomagnesio)-2H-1,3-benzodioxole (1.2)

To a 100 mL RBF was charged Mg (3 g, 149.239 mmol, 2 equiv), 1₂ (315.65 mg, 1.244 mmol, 0.01 equiv) and THF (75 mL) at room temperature under nitrogen. 5-bromo-2H-1,3-benzodioxole (17.5 g, 124.366 mmol, 1 equiv) was added to the mixture and heated to 50° C. at which time the iodine color disappeared, and the internal temperature rose to 56° C. 5-bromo-2H-1,3-benzodioxole (17.5 g, 124.366 mmol, 1 equiv) was added, via syringe, to the mixture dropwise maintaining an internal temperature of 45-55° C. over 10 minutes. After addition was complete the syringe was rinsed with THF (2 mL) and the rinse charged to the reaction at 49° C. After stirring for 1.5 hours the batch was a clear amber color with an internal temperature of 19.6° C. The resulting mixture was used in the next step directly without further purification.

### Step 2. tert-butyl N-[1-(2H-1,3-benzodioxol-5-yl)propan-2-yl]carbamate (1.4)

The flask was cooled to 0.8° C. using an ice/water bath then solid Copper(I) bromide-dimethyl sulfide complex (3.92 g, 19.082 mmol, 0.2 equiv) was charged in one portion. An exotherm to 6° C. was observed. After cooling to 0.5° C. a solution of tert-butyl 2-methylaziridine-1-carboxylate, 1.3 (15 g, 95.412 mmol, 1.00 equiv) was added over 20 minutes, while maintaining an internal temperature <6° C. After stirring for 4 hours TLC analysis (5:1 heptanes/EA) of the brown slurry showed complete reaction. After a further 20 minutes the reaction was quenched with dropwise addition sat. ammonium chloride (500 mL), while maintaining an internal temperature <18° C. (3 minutes). After stirring for 12 minutes at room temperature the biphasic mixture was diluted with EA (100 mL). The layers were separated, and the aqueous layer was extracted with Ethyl acetate (2×100 mL). The combined organic layers dried over sodium sulfate, filtered and concentrated under reduced pressure. Chromatographic purification in silica, eluting with 0-15% EA/PE afforded tert-butyl N-[1-(2H-1,3-benzodioxol-5-yl)propan-2-yl]carbamate (16.7 g) as a white solid. [M+H]⁺ 280.15.

¹H NMR (300 MHz, DMSO-d6) δ 6.79 (d, J=7.9 Hz, 1H), 6.75-6.66 (m, 2H), 6.62 (dd, J=7.9, 1.7 Hz, 1H), 5.94 (q, J=1.0 Hz, 2H), 3.58 (p, J=6.9 Hz, 1H), 2.61 (dd, J=13.4, 7.2 Hz, 1H), 2.46 (d, J=6.7 Hz, 1H), 1.34 (s, 9H), 0.99 (d, J=6.6 Hz, 3H).

### Step 3. 3,4-methylenedioxyamphetamine (1.5)

To a stirred mixture of tert-butyl N-[1-(2H-1,3-benzodioxol-5-yl)propan-2-yl]carbamate (2 g, 7.160 mmol, 1 equiv) in DCM (5 mL) was added HCl(gas) in 1,4-dioxane (15 mL) in portions at 0° C. The resulting mixture was stirred for 0.5 h at 0° C. Desired product could be detected by LCMS. The resulting mixture was concentrated under reduced pressure.

The residue was quenched by the addition of NaHCO₃. The resulting mixture was extracted with EA. The combined organic layers were dried over anhydrous Na₂SO₄. The resulting mixture was concentrated under reduced pressure to afford 3,4-methylenedioxyamphetamine (1.5 g) as a brown oil. [M+H]⁺ 180.09.

### Prodrug 2: N-[1-(2H-1,3-benzodioxol-5-yl)propan-2-yl]methoxycarbothioamide

To a stirred solution of 3,4-methylenedioxyamphetamine (50 mg, 0.279 mmol, 1 equiv) and CS₂ (23.36 mg, 0.307 mmol, 1.1 equiv) in THF (1 mL) was added TEA (56.46 mg, 0.558 mmol, 2 equiv) dropwise at 0° C. under nitrogen atmosphere. The resulting mixture was stirred for 16 h at room temperature under nitrogen atmosphere. MeOH (9.83 mg, 0.307 mmol, 1.1 equiv) was added the above mixture dropwise at room temperature. The resulting mixture was stirred for additional 3 h at room temperature. The reaction was monitored by LCMS. The residue was purified by reversed-phase flash chromatography with the following conditions: column, C18 silica gel; mobile phase, MeCN in Water (0.5% FA), 20% to 40% gradient in 20 min; detector, UV 254 nm. This resulted in N-[1-(2H-1,3-benzodioxol-5-yl)propan-2-yl]methoxycarbothioamide (8.4 mg) as a white solid. [M+H]⁺ 254.08.

¹H NMR (400 MHz, DMSO-d6) δ 8.06 (d, J=7.8 Hz, 1H), 6.80 (d, J=7.8 Hz, 1H), 6.75 (d, J=1.7 Hz, 1H), 6.63 (dd, J=7.9, 1.8 Hz, 1H), 5.96 (s, 2H), 4.03-3.71 (m, 1H), 2.65 (ddt, J=13.4, 7.2, 1.7 Hz, 1H), 2.55 (d, J=6.5 Hz, 1H), 2.17 (t, J=1.2 Hz, 3H), 1.03 (d, J=6.6 Hz, 3H).

### Prodrug 4: N-[1-(2H-1,3-benzodioxol-5-yl)propan-2-yl]ethoxycarbothioamide

To a stirred solution of 3,4-methylenedioxyamphetamine (50 mg, 0.279 mmol, 1 equiv) and CS₂ (23.36 mg, 0.307 mmol, 1.1 equiv) in THF (1 mL) was added TEA (56.46 mg, 0.558 mmol, 2 equiv) dropwise at 0° C. under nitrogen atmosphere. The resulting mixture was stirred for 16 h at room temperature under nitrogen atmosphere. EtOH (14.14 mg, 0.307 mmol, 1.1 equiv) was added the above mixture dropwise at room temperature. The resulting mixture was stirred for additional 3 h at room temperature. The reaction was monitored by LCMS. The residue was purified by reversed-phase Combi-Flash, Column: XBridge Shield RP18 OBD Column, 30*150 mm, 5 μm; Mobile Phase A: Water (10 mmol/L NH₄HCO₃), Mobile Phase B: ACN; Flow rate: 60 mL/min mL/min; Gradient: 47% B to 50% B in 10 min; Wave Length: 254 nm/220 nm nm; RT1(min): 9.1. This resulted in N-[1-(2H-1,3-benzodioxol-5-yl)propan-2-yl]ethoxycarbothioamide (10.9 mg) as a white solid. [M+H]⁺ 268.09.

¹H NMR (400 MHz, Methanol-d4) δ 6.75-6.61 (m, 3H), 5.88 (d, J=0.9 Hz, 2H), 4.46-4.01 (m, 3H), 2.89 (dd, J=13.4, 6.1 Hz, 1H), 2.71-2.57 (m, 1H), 1.25 (dt, J=17.7, 7.1 Hz, 3H), 1.11 (t, J=6.9 Hz, 3H).

### Prodrug 6: N-[1-(2H-1,3-benzodioxol-5-yl)propan-2-yl]methylsulfanylformamide

To a stirred solution of 3,4-methylenedioxyamphetamine (50 mg, 0.279 mmol, 1 equiv) in H₂O (1 mL) was added bis(methylsulfanyl)methanone (68.18 mg, 0.558 mmol, 2 equiv) dropwise at 0° C. under nitrogen atmosphere. The resulting mixture was stirred for 16 h at room temperature under nitrogen atmosphere. The reaction was monitored by LCMS. The residue was purified by reversed-phase Combi-Flash, Column: XBridge Shield RP18 OBD Column, 30*150 mm, 5 μm; Mobile Phase A: Water (0.5% FA), Mobile Phase B: ACN; Flow rate: 60 mL/min mL/min; Gradient: 30% B to 40% B in 10 min; Wave Length: 254 nm/220 nm nm; RT1(min): 9.1. This resulted in N-[1-(2H-1,3-benzodioxol-5-yl) propan-2-yl]methylsulfanylformamide (9.6 mg) as a white solid. [M+H]⁺ 254.08.

¹H NMR (400 MHz, DMSO-d6) δ 8.06 (d, J=7.8 Hz, 1H), 6.90-6.71 (m, 2H), 6.63 (dd, J=8.0, 1.7 Hz, 1H), 5.96 (s, 2H), 3.90 (p, J=7.2 Hz, 1H), 2.65 (dd, J=13.5, 7.3 Hz, 1H), 2.58-2.51 (m, 1H), 2.16 (s, 3H), 1.02 (d, J=6.5 Hz, 3H).

### Prodrug 8: N-[1-(2H-1,3-benzodioxol-5-yl)propan-2-yl]ethylsulfanylformamide

To a stirred solution of 3,4-methylenedioxyamphetamine (50 mg, 0.279 mmol, 1 equiv) and ethanethiol (19.07 mg, 0.307 mmol, 1.1 equiv) in THF (1 mL) was added TEA (56.46 mg, 0.558 mmol, 2 equiv) and ditrichloromethyl carbonate (24.84 mg, 0.084 mmol, 0.3 equiv) dropwise at 0° C. under nitrogen atmosphere. The resulting mixture was stirred for 16 h at room temperature under nitrogen atmosphere. Et₂O (22.75 mg, 0.307 mmol, 1.1 equiv) was added the above mixture dropwise at room temperature. The resulting mixture was stirred for additional 3 h at room temperature. The reaction was monitored by LCMS. The residue was purified by reversed-phase Combi-Flash, Column: XBridge Prep OBD C18 Column, 30*150 mm, 10 μm; Mobile Phase A: Water (10 mmol/L NH₄HCO₃+0.05% NH₃H₂O), Mobile Phase B: ACN; Flow rate: 60 mL/min mL/min; Gradient: 41% B to 48% B in 8 min; Wave Length: 254 nm/220 nm nm; RT1(min): 9.23. This resulted in N-[1-(2H-1,3-benzodioxol-5-yl)propan-2-yl]ethylsulfanylformamide (10.4 mg) as a white solid. [M+H]⁺ 268.09.

¹H NMR (400 MHz, DMSO-d6) δ 7.99 (d, J=7.8 Hz, 1H), 6.80 (d, J=8.0 Hz, 1H), 6.75 (s, 1H), 6.63 (d, J=8.1 Hz, 1H), 5.95 (s, 2H), 3.90 (p, J=6.9 Hz, 1H), 2.73 (q, J=7.3 Hz, 2H), 2.67-2.60 (m, 1H), 2.56-2.52 (m, 1H), 1.14 (t, J=7.3 Hz, 3H), 1.02 (d, J=6.6 Hz, 3H).

### Prodrug 10: N-[1-(2H-1,3-benzodioxol-5-yl) propan-2-yl]methylsulfanylcarbothioamide

To a stirred solution of 3,4-methylenedioxyamphetamine (50 mg, 0.279 mmol, 1 equiv) and CS₂ (23.36 mg, 0.307 mmol, 1.1 equiv) in EtOH (1 mL)/H₂O (0.01 mL) was added TEA (56.46 mg, 0.558 mmol, 2 equiv) dropwise at 0° C. under nitrogen atmosphere. The resulting mixture was stirred for 16 h at room temperature under nitrogen atmosphere. To a stirred solution of CH₃I (43.56 mg, 0.307 mmol, 1.1 equiv) was added above mixture dropwise at room temperature under nitrogen atmosphere. The resulting mixture was stirred for 3 h at room temperature under nitrogen atmosphere. The reaction was monitored by LCMS. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in N-[1-(2H-1,3-benzodioxol-5-yl) propan-2-yl]methylsulfanylcarbothioamide (18.6 mg) as a colorless oil. [M+H]⁺ 270.00 ¹H NMR (400 MHz, DMSO-d6) δ 9.86 (d, J=7.5 Hz, 1H), 6.86-6.75 (m, 2H), 6.68 (dd, J=7.9, 1.7 Hz, 1H), 5.96 (s, 2H), 4.71-4.54 (m, 1H), 2.87 (dd, J=13.5, 6.9 Hz, 1H), 2.62 (dd, J=13.5, 7.0 Hz, 1H), 2.52-2.50 (m, 2H), 1.34 (s, 1H), 1.11 (d, J=6.6 Hz, 3H).

### Prodrug 12: N-[1-(2H-1,3-benzodioxol-5-yl) propan-2-yl]ethylsulfanylcarbothioamide

To a stirred solution of 3,4-methylenedioxyamphetamine (50 mg, 0.279 mmol, 1 equiv) and CS₂ (23.36 mg, 0.307 mmol, 1.1 equiv) in EtOH (1 mL) was added TEA (56.46 mg, 0.558 mmol, 2 equiv) dropwise at 0° C. under nitrogen atmosphere. The resulting mixture was stirred for 16 h at room temperature under nitrogen atmosphere. iodoethane (43.56 mg, 0.307 mmol, 1.1 equiv) was added the above mixture dropwise at room temperature. The resulting mixture was stirred for additional 3 h at room temperature. The reaction was monitored by LCMS. The residue was purified by reversed-phase flash chromatography with the following conditions: column, C18 silica gel; mobile phase, MeCN in Water (10 mmol/L NH₄HCO₃), 10% to 70% gradient in 10 min; detector, UV 254 nm. This resulted in N-[1-(2H-1,3-benzodioxol-5-yl) propan-2-yl]ethylsulfanylcarbothioamide (11.3 mg) as a white solid. [M+H]⁺ 283.95 ¹H NMR (400 MHz, DMSO-d6) δ 9.83 (d, J=7.6 Hz, 1H), 6.91-6.75 (m, 2H), 6.67 (dd, J=7.9, 1.7 Hz, 1H), 5.96 (d, J=1.1 Hz, 2H), 4.62 (p, J=6.9 Hz, 1H), 3.13 (qd, J=7.3, 4.8 Hz, 2H), 2.86 (dd, J=13.4, 6.7 Hz, 1H), 2.62 (dd, J=13.5, 7.0 Hz, 1H), 1.20 (t, J=7.3 Hz, 3H), 1.11 (d, J=6.6 Hz, 3H).

These prodrugs were made via following three steps (scheme 2).

### Synthesis of Representative Compounds, 1.0, 3.0, 5.0, 7.0, 9.0 and 11.

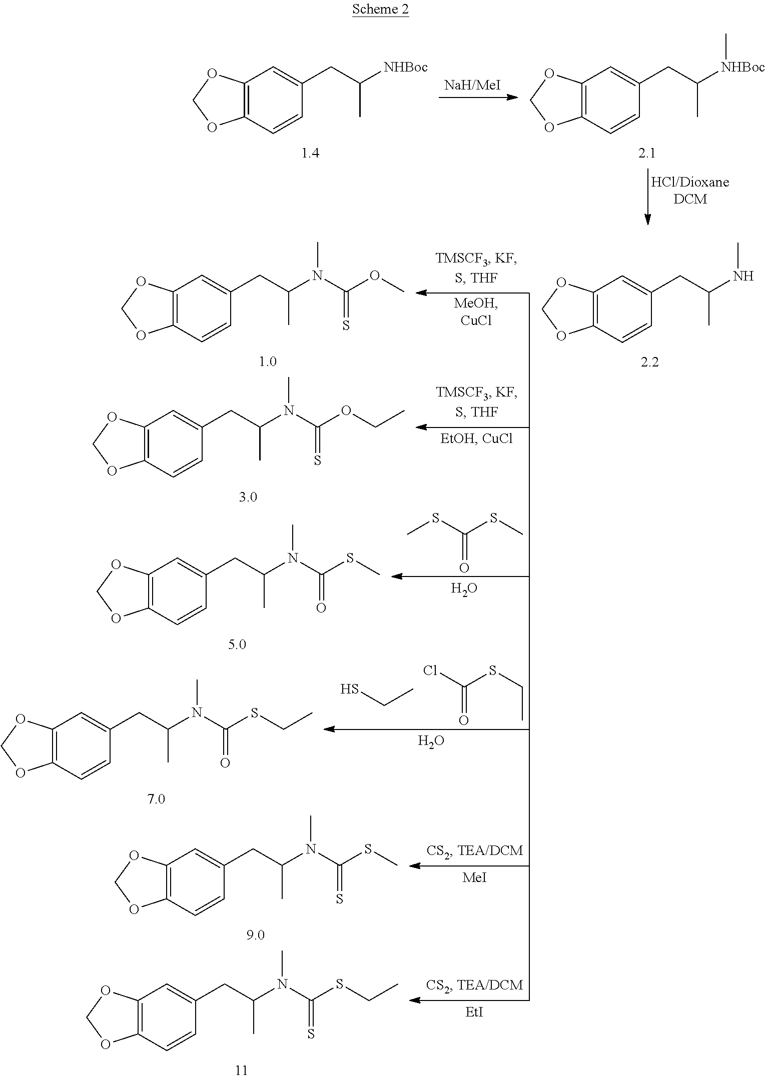

### Synthesis of Intermediate 2.2.

### Step 1: Tert-butyl (1-(benzo[d][1,3]dioxol-5-yl) propan-2-yl) (methyl)carbamate (2.1)

To a stirred mixture of tert-butyl N-[1-(2H-1,3-benzodioxol-5-yl) propan-2-yl]carbamate, 1.4 (5 g, 17.900 mmol, 1 equiv) in DMF (50 mL) was added NaH (0.64 g, 26.850 mmol, 1.5 equiv) in portions at 0° C. The resulting mixture was stirred for 0.5 h at 0° C. And then add Mel (3.81 g, 26.850 mmol, 1.5 equiv). The resulting mixture was stirred for 2 h at 25° C. The reaction was monitored by LCMS. The reaction was quenched by the addition of water (10 mL). The resulting mixture was extracted with EA. The combined organic layers were dried over anhydrous Na₂SO₄. After filtration, the filtrate was concentrated under reduced pressure. The crude product was used in the next step directly without further purification.

### Step 2: 1-(benzo[d][1,3]dioxol-5-yl)-N-methylpropan-2-amine (2.2)

To a stirred mixture of tert-butyl N-[1-(2H-1,3-benzodioxol-5-yl) propan-2-yl]-N-methylcarbamate 2.1 (3 g, 10.226 mmol, 1 equiv) in DCM (7 mL) was added HCl(gas) in 1,4-dioxane (21 mL) in portions at 0° C. The resulting mixture was stirred for 0.5 h at 0° C. The reaction was monitored by LCMS. The resulting mixture was concentrated under reduced pressure to afford MDMA (1.5 g) as a white solid. [M+H]⁺ 194.0

### Prodrug 1.0: N-[1-(2H-1,3-benzodioxol-5-yl)propan-2-yl]-N-methylmethoxy-carbothioamide

To a stirred solution of MDMA (100 mg, 0.517 mmol, 1 equiv) and TMSCF₃ (80.94 mg, 0.569 mmol, 1.1 equiv) in THF (1 mL) were added KF (60.13 mg, 1.034 mmol, 2 equiv) and S (24.84 mg, 0.776 mmol, 1.5 equiv) in portions at room temperature under nitrogen atmosphere. The resulting mixture was stirred at room temperature for 2 h under nitrogen atmosphere. To the above mixture was added CuCl (10.25 mg, 0.103 mmol, 0.2 equiv) and MeOH (1 mL) in portions. The resulting mixture was stirred at 50° C. for additional 1 h. Desired product could be detected by LCMS. The residue was purified by reversed-phase flash chromatography with the following conditions: Column: Xbridge Phenyl OBD Column, 30*150 mm, 5 m; Mobile Phase A: Water (10 mmol/L NH₄HCO₃, Mobile Phase B: ACN; Gradient: 44% B to 45% B in 10 min; Wave Length: 254 nmnm. The resulting mixture was concentrated under reduced pressure to afford N-[1-(2H-1,3-benzodioxol-5-yl)propan-2-yl]-N-methyl-methoxycarbothioamide (17.9 mg) as a light yellow oil. [M+H]⁺ 268.05.

¹H NMR (400 MHz, DMSO-d₆) δ 6.86-6.70 (m, 2H), 6.64 (s, 1H), 5.96 (dd, J=4.0, 1.1 Hz, 2H), 4.35 (d, J=215.5 Hz, 1H), 2.78 (s, 3H), 2.68 (d, J=10.9 Hz, 2H), 2.16 (s, 3H), 1.10 (d, J=17.3 Hz, 3H).

### Prodrug 3.0: N-[1-(2H-1,3-benzodioxol-5-yl)propan-2-yl]-N-methylethoxycarbothioamide

To a stirred solution of MDMA (100 mg, 0.517 mmol, 1 equiv) and TMSCF3 (80.94 mg, 0.569 mmol, 1.1 equiv) in THF (1 mL) were added KF (60.13 mg, 1.034 mmol, 2 equiv) and S (24.84 mg, 0.776 mmol, 1.5 equiv) in portions at room temperature under nitrogen atmosphere. The resulting mixture was stirred at room temperature for 2 h under nitrogen atmosphere. To the above mixture was added CuCl (10.25 mg, 0.103 mmol, 0.2 equiv) and EtOH (1 mL) in portions. The resulting mixture was stirred at 50° C. for additional 1 h. Desired product could be detected by LCMS. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 220 nm. The resulting mixture was concentrated under reduced pressure to afford N-[1-(2H-1,3-benzodioxol-5-yl)propan-2-yl]-N-methylethoxycarbothioamide (12.7 mg) as a white solid. [M+H]⁺ 281.95.

¹H NMR (400 MHz, DMSO-d₆) δ 6.84-6.71 (m, 2H), 6.64 (ddd, J=33.4, 7.9, 1.7 Hz, 1H), 6.02-5.87 (m, 2H), 5.09 (dq, J=224.5, 7.1 Hz, 1H), 4.37 (qt, J=7.0, 3.6 Hz, 1H), 4.30-4.19 (m, 1H), 2.98 (d, J=84.3 Hz, 3H), 2.81-2.63 (m, 2H), 1.28-1.04 (m, 6H).

### Prodrug 5.0: N-[1-(2H-1,3-benzodioxol-5-yl)propan-2-yl]-N-methylmethylsulfanylformamide

To a stirred solution of MDMA (100 mg, 0.517 mmol, 1 equiv) in H₂O (1 mL) was added bis(methylsulfanyl)methanone (126.47 mg, 1.034 mmol, 2 equiv) in portions at 0° C. under nitrogen atmosphere. The resulting mixture was stirred at room temperature for overnight under nitrogen atmosphere. Desired product could be detected by LCMS. The residue was purified by reversed-phase flash chromatography with the following conditions: Column: Xbridge Phenyl OBD Column, 30*150 mm, 5 m; Mobile Phase A: Water (10 mmol/L NH₄HCO₃, Mobile Phase B: ACN; Gradient: 44% B to 45% B in 10 min; Wave Length: 254 nm/220 nm. The resulting mixture was concentrated under reduced pressure to afford N-[1-(2H-1,3-benzodioxol-5-yl)propan-2-yl]-N-methylmethylsulfanylformamide (6.3 mg) as a light yellow oil. [M+H]⁺ 268.05.

¹H NMR (400 MHz, DMSO-d₆) δ 6.86-6.70 (m, 2H), 6.64 (s, 1H), 6.03-5.89 (m, 2H), 4.35 (d, J=215.3 Hz, 1H), 2.78 (s, 3H), 2.75-2.62 (m, 2H), 2.16 (s, 3H), 1.10 (d, J=17.2 Hz, 3H).

### Prodrug 7.0: N-[1-(2H-1,3-benzodioxol-5-yl)propan-2-yl]-N-methylethylsulfanylformamide

To a stirred solution of MDMA (100 mg, 0.517 mmol, 1 equiv) in H₂O (1 mL) was added S-ethyl chlorothiolformate (128.93 mg, 1.034 mmol, 2 equiv) in portions at 0° C. under nitrogen atmosphere. The resulting mixture was stirred at room temperature for overnight under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 66% gradient in 30 min; detector, UV 220 nm. The resulting mixture was concentrated under reduced pressure to afford N-[1-(2H-1,3-benzodioxol-5-yl)propan-2-yl]-N-methylethylsulfanylformamide (29.2 mg) as a colorless oil. [M+H]⁺ 268.09.

¹H NMR (400 MHz, DMSO-d₆) δ 6.87-6.69 (m, 2H), 6.62 (d, J=9.5 Hz, 1H), 5.95 (d, J=2.1 Hz, 2H), 4.36 (d, J=219.5 Hz, 1H), 2.71 (dd, J=14.1, 6.8 Hz, 7H), 1.10 (q, J=6.5, 5.6 Hz, 6H).

### Prodrug 9.0: N-[1-(2H-1,3-benzodioxol-5-yl) propan-2-yl]-N-methylmethylsulfanyl-carbothioamide

To a stirred solution of MDMA (50 mg, 0.259 mmol, 1 equiv) and CS₂ (21.67 mg, 0.285 mmol, 1.1 equiv) in EtOH (1 mL)/H₂O (0.01 mL) was added TEA (52.36 mg, 0.518 mmol, 2 equiv) dropwise at 0° C. under nitrogen atmosphere. The resulting mixture was stirred for 16 h at room temperature under nitrogen atmosphere. To a stirred solution of CH₃I (40.40 mg, 0.285 mmol, 1.1 equiv) was added above mixture dropwise at room temperature under nitrogen atmosphere. The resulting mixture was stirred for 3 h at room temperature under nitrogen atmosphere. The reaction was monitored by LCMS. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in N-[1-(2H-1,3-benzodioxol-5-yl) propan-2-yl]-N-methylmethylsulfanyl-carbothioamide (8.6 mg) as a white solid. [M+H]⁺ 284.05

¹H NMR (400 MHz, DMSO-d6) δ 6.91-6.77 (m, 2H), 6.68 (dt, J=7.9, 2.0 Hz, 1H), 6.04-5.94 (m, 2H), 5.92-4.77 (m, 1H), 3.29 (s, 1H), 3.15 (s, 2H), 2.88-2.71 (m, 2H), 2.53 (s, 2H), 1.21 (d, J=6.5 Hz, 1H), 1.11 (d, J=6.7 Hz, 2H).

### Prodrug 11: N-[1-(2H-1,3-benzodioxol-5-yl) propan-2-yl]-N-methylethylsulfanylcarbothioamide

To a stirred solution of MDMA (50 mg, 0.259 mmol, 1 equiv) and CS₂ (21.67 mg, 0.285 mmol, 1.1 equiv) in EtOH (1 mL)/H₂O (0.01 mL) was added TEA (52.36 mg, 0.518 mmol, 2 equiv) dropwise at 0° C. under nitrogen atmosphere. The resulting mixture was stirred for 16 h at room temperature under nitrogen atmosphere. To a stirred solution of iodoethane (44.39 mL, 0.285 mmol, 1.1 equiv) was added the above mixture dropwise at room temperature under nitrogen atmosphere. The resulting mixture was stirred for 3 h at room temperature under nitrogen atmosphere. The reaction was monitored by LCMS. The residue was purified by reversed-phase flash chromatography with the following conditions: column, C18 silica gel; mobile phase, MeCN in Water (10 mmol/L NH₄HCO₃), 10% to 70% gradient in 10 min; detector, UV 254 nm. This resulted in N-[1-(2H-1,3-benzodioxol-5-yl) propan-2-yl]-N-methylethylsulfanylcarbothioamide (10.8 mg) as a colorless oil. [M+H]⁺ 298.05 ¹H NMR (400 MHz, DMSO-d6) δ 6.86-6.75 (m, 2H), 6.67 (dt, J=7.9, 2.0 Hz, 1H), 6.01-5.93 (m, 2H), 5.37 (dp, J=399.0, 7.0 Hz, 1H), 3.28 (s, 1H), 3.22-3.10 (m, 4H), 2.86-2.71 (m, 2H), 1.27-1.14 (m, 4H), 1.11 (d, J=6.7 Hz, 2H).

### Stability of Prodrugs:

### Chemical Stability:

12 prodrugs Table 1. Stability results of test compounds and control compound in PBS pH 6.5

TABLE 1

| | | Remaining Percentage (%) | | | | |
|---|---|---|---|---|---|---|
| | T_{1/2} | 0 | 30 | 60 | 120 | 240 |
| Prodrugs | (min) | min | min | min | min | min |
| Diclofenac | 41.68 | 100.00 | 60.68 | 35.44 | 13.15 | 1.85 |
| 7 | >1023 | 100.00 | 99.17 | 97.73 | 95.86 | 103.73 |
| 1 | >1023 | 100.00 | 95.62 | 102.35 | 93.11 | 106.73 |
| 5 | >1023 | 100.00 | 91.67 | 104.03 | 96.42 | 91.92 |
| 3 | >1023 | 100.00 | 100.73 | 95.33 | 99.99 | 91.16 |
| 6 | >1023 | 100.00 | 93.08 | 91.55 | 88.38 | 88.38 |
| 4 | >1023 | 100.00 | 103.44 | 100.85 | 94.76 | 94.28 |
| 2 | >1023 | 100.00 | 105.00 | 100.45 | 102.27 | 105.00 |
| 8 | >1023 | 100.00 | 113.02 | 100.82 | 102.82 | 114.28 |
| 10 | >1023 | 100.00 | 96.55 | 105.17 | 109.86 | 113.62 |
| 11 | >1023 | 100.00 | 95.01 | 93.41 | 96.45 | 94.38 |
| 9 | >1023 | 100.00 | 97.22 | 96.84 | 107.55 | 89.55 |
| 12 | >1023 | 100.00 | 102.94 | 99.30 | 104.13 | 100.98 |

Note:
If % remaining at 240 minutes was greater than 85%, then t½ will be reported as “> 1023.39

### Solubility: Table 2. Solubility Result of Test Compounds and Control Compound in FaSSIF

TABLE 2

| | SOL (μM) |
|---|---|
| Compound ID | FaSSIF |
| Diclofenac | 291.73 |
| 7 | 291.29 |
| 1 | 278.19 |
| 5 | 307.73 |
| 3 | 131.06 |
| 6 | 292.68 |
| 4 | 60.34 |
| 2 | 279.33 |
| 8 | 288.40 |
| 10 | 306.20 |
| 11 | 63.72 |
| 9 | 30.45 |
| 12 | 251.50 |

Any value close to or above 300 μM indicates that the compound may have a solubility at or above 300 μM.

### Plasma Stability

### Table 3. Plasma Stability in Mouse of the 12 Compounds and Control Compound Propantheline in Mouse Plasma.

TABLE 3

| | Test | | Remaining Percentages (%) | | | | |
|---|---|---|---|---|---|---|---|
| | concentration | T_{1/2} | 0 | 15 | 30 | 60 | 120 |
| Prodrugs | (μM) | (min) | min | min | min | min | min |
| Diclofenac | 5 | 35.61 | 100 | 85.27 | 58.97 | 29.25 | 10.45 |
| 7 | 1 | >511 | 100 | 110.62 | 111.95 | 113.27 | 102.65 |
| 1 | 1 | >511 | 100 | 108.17 | 96.20 | 103.37 | 100.00 |
| 5 | 1 | >511 | 100 | 100.78 | 98.96 | 100.78 | 94.52 |
| 3 | 1 | >511 | 100 | 106.31 | 97.30 | 97.75 | 96.85 |
| 6 | 1 | >511 | 100 | 99.73 | 103.77 | 102.16 | 90.30 |
| 4 | 1 | >511 | 100 | 100.00 | 100.28 | 97.80 | 95.32 |
| 2 | 1 | >511 | 100 | 101.54 | 99.62 | 96.35 | 91.94 |
| 8 | 1 | >511 | 100 | 110.30 | 106.34 | 105.81 | 101.19 |
| 10 | 1 | >511 | 100 | 100.89 | 98.00 | 100.22 | 89.09 |
| 11 | 1 | >511 | 100 | 97.92 | 101.25 | 94.58 | 94.17 |
| 9 | 1 | >511 | 100 | 101.32 | 96.64 | 98.79 | 97.14 |
| 12 | 1 | >511 | 100 | 101.19 | 92.04 | 92.28 | 89.25 |
| 7 | 1 | >511 | 100 | 110.62 | 111.95 | 113.27 | 102.65 |

Note:
when the remaining percentage of the last time point is > 85%, T_{1/2} (min) is reported as > 511.69 min

1. A compound of Formula (VI): 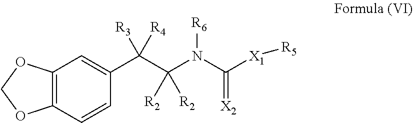
wherein X₁ and X₂ are independently O, S, or N;
R₁, R₂, R₃, and R₄ are independently hydrogen, deuterium, alkyl, deuterated alkyl, CH₂D, CHD₂, CD₃, cycloalkyl, halogen, heteroatom-substituted or halogen-substituted alkyl or cycloalkyl, aryl, or heteroaryl; and
R₅ and R₆, are independently hydrogen, deuterium, CH₂D, CHD₂, CD₃, or a C₁ to C₆ alkyl.

2. The compound of claim 1, wherein X₂ is S.

3. The compound of claim 2, wherein X₁ is O.

4. The compound of claim 2, wherein X₁ is S.

5. The compound of claim 1, wherein R₂, R₃, R₄, and R₆ are hydrogen or deuterium.

6. The compound of claim 5, wherein R₁ is methyl.

7. The compound of claim 6, wherein R₅ is methyl.

8. The compound of claim 5, wherein R₁ is ethyl.

9. The compound of claim 5, wherein R₁ is propyl.

10. The compound of claim 9, wherein R₁ is isopropyl.

11. The compound of claim 5, wherein R₅ is ethyl.

12. The compound of claim 1, wherein R₆ is methyl.

13. The compound of claim 1, wherein R₁ and R₂ combine to form a 3-6 membered cycloalkyl.

14. The compound of claim 13, wherein R₁ and R₂ combine to form a 3 membered ring.

15. The compound of claim 13, wherein R₁ and R₂ combine to form a 4 membered ring.

16. The compound of claim 13, wherein R₁ and R₂ combine to form a 5 membered ring.

17. The compound of claim 13, wherein R₁ and R₂ combine to form a 6 membered ring.

18. The compound of claim 1, wherein R₁ is a halogenated alkyl.

19. The compound of claim 1, wherein R₃ and R₄ are halogens.

20. The compound of claim 1, wherein R₁ is an ether.

21. The compound of claim 1, wherein the compound is an R enantiomer.

22. The compound of claim 1, wherein the compound is an S enantiomer.

23. The compound of claim 1, wherein the compound persists through a first pass metabolism of a mammal.

24. The compound of claim 23, wherein the compound acts as a partial agonist of a CNS serotonin receptor.

25. A composition comprising a pharmaceutical carrier and a compound of Formula (VI): 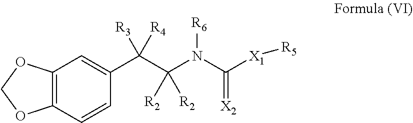
wherein X₁ and X₂ are independently O, S, or N;
R₁, R₂, R₃, and R₄ are independently hydrogen, deuterium, alkyl, deuterated alkyl, CH₂D, CHD₂, CD₃, cycloalkyl, halogen, heteroatom-substituted or halogen-substituted alkyl or cycloalkyl, aryl, or heteroaryl; and
R₅ and R₆, are independently hydrogen, deuterium, methyl, ethyl, CH₂D, CHD₂, CD₃, or a C₁ to C₆ alkyl.

26. The composition of claim 25, wherein X₂ is S.

27. The composition of claim 26, wherein X₁ is O.

28. The composition of claim 26, wherein X₁ is S.

29. The composition of claim 25, wherein R₂, R₃, R₄, and R₆ are hydrogen or deuterium.

30. The composition of claim 29, wherein R₁ is methyl.

31. The composition of claim 30, wherein R₅ is methyl.

32. The composition of claim 29, wherein R₁ is ethyl.

33. The composition of claim 29, wherein R₁ is propyl.

34. The composition of claim 33, wherein R₁ is isopropyl.

35. The composition of claim 25, wherein R₅ is ethyl.

36. The composition of claim 25, wherein R₆ is methyl.

37. The composition of claim 25, wherein R₁ and R₂ combine to form a 3-6 membered cycloalkyl.

38. The composition of claim 37, wherein R₁ and R₂ combine to form a 3 membered ring.

39. The composition of claim 37, wherein R₁ and R₂ combine to form a 4 membered ring.

40. The composition of claim 37, wherein R₁ and R₂ combine to form a 5 membered ring.

41. The composition of claim 37, wherein R₁ and R₂ combine to form a 6 membered ring.

42. The composition of claim 25, wherein R₁ is a halogenated alkyl.

43. The composition of claim 25, wherein R₃ and R₄ are halogens.

44. The composition of claim 25, wherein R₁ is an ether.

45. The composition of claim 25, wherein the compound is an R enantiomer.

46. The composition of claim 25, wherein the compound is an S enantiomer.

47. The composition of claim 25, wherein the compound persists through a first pass metabolism of a mammal.

48. The composition of claim 47, wherein the compound acts as a partial agonist of a CNS serotonin receptor.

49. The composition of claim 25, wherein the pharmaceutical carrier is a bulking agent.
